(19) United States
(12) Patent Application Publication
(10) Pub. No.: US 2022/0348866 A1
(43) Pub. Date: Nov. 3, 2022

(54) MODIFIED INDUCED PLURIPOTENT STEM CELL (iPSC) DERIVED MICROGLIA FOR THE TREATMENT OF BRAIN CANCER

(71) Applicant: Vala Sciences Inc., San Diego, CA (US)

(72) Inventors: Patrick M. McDonough, San Diego, CA (US); Jeffrey H. Price, San Diego, CA (US); Daniel R. Rines, San Diego, CA (US); Christine Gjerdrum Rines, San Diego, CA (US); Ranor C. B. Basa, San Diego, CA (US)

(21) Appl. No.: 17/720,753

(22) Filed: Apr. 14, 2022

Related U.S. Application Data
(60) Provisional application No. 63/181,600, filed on Apr. 29, 2021.

(51) Int. Cl. C12N 5/079; A61K 35/30

(52) U.S. Cl. CPC C12N 5/0622; A61K 35/30; C12N 2506/45

(57) ABSTRACT

The present disclosure provides modified induced pluripotent stem cell (iPSC) derived microglia comprising a microglia targeting and activation protein, a microglia regulatory protein, an interfering RNA sequence, a microRNA effector, a non-coding RNA effector, at least one transgene encoding the aforementioned, or a combination thereof. Also provided are methods for treating cancer (e.g., brain cancers) and sensitizing cancer cells to phagocytosis and other cell death pathways with the modified induced pluripotent stem cell (iPSC) derived microglia.

### STATEMENT REGARDING RELATED APPLICATIONS

This application claims priority to U.S. Provisional Patent Application No. 63/181,600, filed Apr. 29, 2021, the entire contents of which are incorporated herein by reference for all purposes.

### FIELD

The present disclosure provides modified induced pluripotent stem cell (iPSC) derived microglia for treating brain cancers.

### BACKGROUND

Glioblastoma (GB) is the most common lethal brain tumor. Those diagnosed with GB generally succumb to the disease in a matter of months (median survival ˜15 months, 5-year survival rates <5%). (CBTRUS; cbtrus.org)). Although some progress has been made in the treatment of glioblastoma, this disease presents a highly unmet medical need with limited treatment options. The current GB standard of care is surgery to remove or resect the GB tumor(s) and follow with radiation and chemotherapy with the DNA alkylating agent temozolomide, although this agent only extends life by a few months vs. radiation alone.

### SUMMARY

Disclosed herein are induced pluripotent stem cell derived microglia comprising one or more or a) a microglia targeting and activation protein, or a nucleic acid encoding a microglia targeting and activation protein, wherein the microglia targeting and activation protein comprises an extracellular domain comprising a leader sequence, one or more target-recognition domains, a spacer sequence, a transmembrane region, and a cytoplasmic region comprising one or more domains from regulatory proteins that activate microglial cells to engulf or kill target cells; b) a modified microglia regulatory protein, or a nucleic acid encoding a modified microglia regulatory protein; c) an interfering RNA sequence, or a nucleic acid encoding an interfering RNA sequence; d) a microRNA effector, an entity that either mimics or antagonizes the action of a microRNA, or a nucleic acid encoding a microRNA effector; and e) a non-coding RNA effector, or a nucleic acid encoding a non-coding RNA effector.

Also disclosed herein is a method of manufacturing the induced pluripotent stem cell derived microglia comprising acquiring an induced pluripotent stem cell derived microglial cell and introducing one or more of a microglia targeting and activation protein, a modified microglia regulatory protein, an interfering RNA sequence, a microRNA effector, a non-coding RNA effector, at least one nucleic acid encoding thereof, or a combination thereof into the induced pluripotent stem cell derived microglial cell.

Further disclosed are methods of using the induced pluripotent stem cell derived microglia. In some embodiments, the methods comprise killing one or more cancer cells comprising contacting cancer cells with the induced pluripotent stem cell derived microglia described herein. In some embodiments, the methods comprise treating cancer in a subject comprising administering to the subject a pharmaceutical composition comprising a population of the induced pluripotent stem cell derived microglia described herein. In some embodiments, the cancer is brain cancer. In some embodiments, the cancer is glioblastoma. In some embodiments, the cancer is located in the spinal cord.

Other aspects and embodiments of the disclosure will be apparent in light of the following detailed description and accompanying figures.

### BRIEF DESCRIPTION OF DRAWINGS

FIG. 1 is a schematic of an exemplary design of microglial targeting and activation proteins (MTAPs).

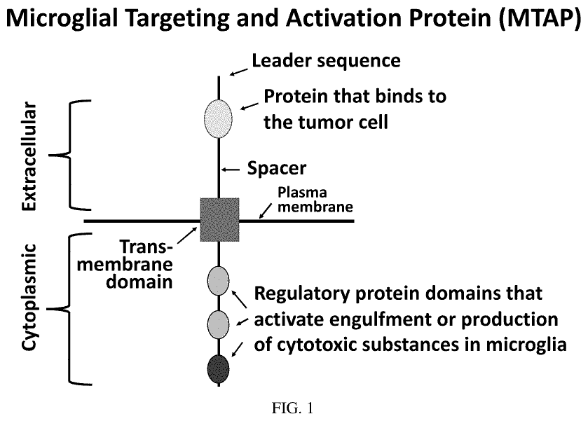

### DETAILED DESCRIPTION

CAR-T strategies have been tested to treat glioblastoma and many clinical trials are underway. While these strategies have proven relatively safe, most have yielded inconclusive, or at best, partial responses. Promising results using CAR-T have indicated that immunotherapy is a viable approach to treating glioblastoma but the strategy has as yet demonstrated only marginal success and has not yet achieved FDA approval. A key problem is that T-cells do not readily migrate into glioblastoma tumors, which severely limits their access to the glioblastoma cells, and this and other factors (e.g., immuno-evasive mechanisms employed by the tumor cells) may decrease their ability to destroy glioblastoma tumor cells. The disclosed modified induced pluripotent stem cell (iPSC) derived microglia improve treatment of brain cancers by increasing penetration of the tumor and expressing one or more transgenes which enhance the ability of the cells to destroy tumor cells, and/or to recruit other components of immune system which destroy tumor cells.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

A “chemotherapeutic agent,” as used herein, refers to a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinas inhibitors. Chemotherapeutic agents include compounds used in “targeted therapy” and conventional chemotherapy. Examples of chemotherapeutic agents include, but are not limited to: erlotinib, docetaxel, 5-FU, gemcitabine, PD-0325901, cisplatin, carboplatin, paclitaxel, trastuzumab, temozolomide, doxorubicin, Akti-1/2, HPPD, and rapamycin.

The term “contacting” as used herein refers to bring or put in contact, to be in or come into contact. The term “contact” as used herein refers to a state or condition of touching or of immediate or local proximity Contacting a composition to a target destination, such as, but not limited to, an organ, tissue, cell, or tumor, may occur by any means of administration known to the skilled artisan.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by introducing certain factors, referred to as reprogramming factors.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing,” “administering,” and “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

As used herein, “treat,” “treating” and the like means a slowing, stopping, or reversing of progression of a disease or disorder when provided a peptide or composition described herein to an appropriate subject. The term also includes a reversing of the progression of such a disease or disorder to a point of eliminating or greatly reducing the disease. As such, “treating” means an application or administration of the peptides or compositions described herein to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Induced Pluripotent Stem Cell Derived Microglia

The present application discloses induced pluripotent stem cell derived microglia comprising one or more transgenes or products thereof. In some embodiments, the induced pluripotent stem cell derived microglia comprising one or more transgenes or products thereof may target (e.g., activate and/or optimize) various pathways within the cells as shown in Table 1.

TABLE 1
Pathways

| | |
|---|---|
| 1. Phagocytosis | A classical pathway for engulfment of debris |
| (engulfment) and | and pathogenic cells. |
| trogocytosis | |
| 2. Production of | Proteins which kill targeted cells expressed at the |
| membrane-bound | plasma membrane, or released into the extra- |
| and/or soluble | cellular milieu. Examples include members |
| cytotoxic substances | of the TNFalpha superfamily, and FasL. |
| 3. Antigen | Presentation of antigens to CD8+ T cells via the |
| presentation | MHC Class I pathway, or to CD4+ T cells |
| | via the MHC Class II pathway, leading to |
| | increased recognition of tumor cells by the |
| | adaptive immune system. |
| 4. Interference with | Tumor cells often express proteins (e.g., |
| pathways used | CD47) which enable evasion of the host |
| by tumor cells to | immune system. Expression of appropriate |
| evade the host | transgenes may counteract these evasive |
| immune system | mechanisms. |

In some embodiments, the induced pluripotent stem cell derived microglia comprise one or more of:

a) a microglia targeting and activation protein, or a nucleic acid encoding a microglia targeting and activation protein, wherein the microglia targeting and activation protein comprises an extracellular domain comprising a leader sequence, one or more target-recognition domains, a spacer sequence, a transmembrane region, and a cytoplasmic region comprising one or more domains from regulatory proteins that activate microglial cells to engulf or kill target cells;

b) a modified microglia regulatory protein, or a nucleic acid encoding a modified microglia regulatory protein;

c) an interfering RNA sequence, or a nucleic acid encoding an interfering RNA sequence;

d) a microRNA effector, or a nucleic acid encoding a microRNA effector; and

e) a non-coding RNA effector, or a nucleic acid encoding a non-coding RNA effector.

In some embodiments, the induced pluripotent stem cell derived microglia comprises a microglia targeting and activation protein, or a nucleic acid encoding a microglia targeting and activation protein. The microglia targeting and activation protein (MTAP) comprises an extracellular domain comprising a leader sequence, one or more target-recognition domains, a spacer sequence, a transmembrane region, and a cytoplasmic region comprising one or more domains from regulatory proteins that activate microglial cells to engulf or kill target cells. Unlike chimeric antigen receptors (CARs) which are designed to efficiently work with T cells, MTAPs are specifically designed to be expressed at the plasma membrane and function in microglia. For example, MTAPs can be constructed to express immune receptor tyrosine-based activation motif (ITAM) from DAP12, CD32a (FcgRIIA), and CD32c (FcgRIIC) in the cytoplasmic domain. These proteins are expressed by microglia and are associated with activation of phagocytosis by microglia (See, for example, Linnartz, B., Wang, Y. & Neumann, H. Int J Alzheimers Dis 2010(2010), incorporated herein by reference in its entirety).

The MTAP can be configured to target glioblastoma as well as other tumor or cancerous cells by utilizing protein domains in the extracellular region of the MTAPs that will bind to proteins specifically expressed by other tumor types.

In some embodiments, the induced pluripotent stem cell derived microglia comprises a modified microglia regulatory protein, or a nucleic acid encoding a modified microglia regulatory protein. The modified microglia regulatory protein may enhance targeting to tumor cells, engulfment or killing of tumor cells, or a combination thereof. The modified microglia regulatory protein may increase antigen presentation of the induced pluripotent stem cell derived microglia. The protein may be an optimized endogenous protein having mutations, deletions, or addition of functional groups which confers enhancement of targeting, engulfment, or killing or tumor cells or increase antigen presentation of the induced pluripotent stem cell derived microglia. For example, the protein may be phosphatase and tensin homolog (PTEN), which, when mutated to remove nuclear localization sequences, becomes more cytoplasmic in location and enhances phagocytotic activity of microglia. See, for example, Sam, N., et al. Molecular psychiatry (2020) , incorporated herein by reference in its entirety. A knockout of NPC1 enhances phagocytosis by microglia (Colombo, A., et al. Nature communications 12, 1158 (2021), incorporated herein by reference in its entirety). Thus, genetic editing methods may be used to modify PTEN or inhibit NPC2 expression to increase phagocytotic activity of induced pluripotent stem cell derived microglia.

In some embodiments, the induced pluripotent stem cell derived microglia comprises an interfering RNA sequence (e.g., siRNA or related RNA species), or a nucleic acid encoding an interfering RNA sequence. In some embodiments, the interfering RNA sequence is configured to down regulate proteins which results in an increase in recognition, engulfment, and/or killing of tumor cells. An exemplary interfering RNA sequence comprises an siRNA targeting signal regulatory protein alpha (aSIRP), which recognizes CD47 as a “don't eat me” signal. Another potential target for an siRNA would be SHP-1, a protein tyrosine phosphatase associated with inhibiting microglial activation. Expressing an siRNA against SHP-1 may increase the ability of induced pluripotent stem cell derived microglia to phagocytose or otherwise kill tumor cells.

In some embodiments, the induced pluripotent stem cell derived microglia comprises a microRNA effector, or a nucleic acid encoding a microRNA effector. The microRNA effector may enhance targeting to tumor cells, the engulfment or killing of tumor cells, or a combination thereof. In some embodiments, the microRNA effector is an antagonist of endogenous microRNA effectors (antagomirs [antagonists of miRs], miR decoys, or miR sponges). One example of a microRNA that inhibits phagocytosis is miR-124-5p (Herdoiza Padilla, E., et al. Frontiers in immunology 10, 2210 (2019), incorporated herein by reference in its entirety). Thus, an antagomir of miR-124-5p may enhance engulfment of tumor cells by hiPSC-MG. Another example of a microRNA that enhances phagocytosis is miR-146a (Cao, Z., Yao, Q. & Zhang, S. American journal of translational research 7, 1467-1474 (2015), incorporated herein by reference in its entirety). Thus, a miR-146a mimic could increase the ability of the hiPSC-MG to engulf tumor cells.

In some embodiments, the induced pluripotent stem cell derived microglia comprises a non-coding RNA effector, or a nucleic acid encoding a non-coding RNA effector. Non-coding RNA (ncRNA) effectors are RNA species that do not code for proteins but which regulate expression of both protein and RNA genes throughout the human genome. Genes for ncRNA out number genes that encode proteins in the human genome, and many of these have not yet been assigned function. Classes included in ncRNA include the long ncRNAs (lncRNA, which are important regulators of immune cell function), piwi-interacting RNAs, tRNA fragments, and small nucleolar RNAs (snoRNAs). An example of a ncRNA that regulates microglia is LncRNA-1810034E14Rik. Overexpression of LncRNA-1810034E14Rik reduces activation of microglia (Zhang, X., et al. Journal of neuroinflammation 16, 75 (2019), incorporated herein by reference in its entirety). An effector that antagonizes LncRNA-1810034E14Rik would likely increase the activity of hiPSC-MG to engulf or otherwise kill tumor cells. Similarly, lnc:RNA NEAT1 inhibits transition of microglia towards an inflammatory phenotype (Ni, X., et al. Scientific reports 10, 19658 (2020), incorporated herein by reference in its entirety). Thus, an effector that antagonizes 1ncRNA NEAT1 would likely increase the activity of hiPSC-MG to engulf or otherwise kill tumor cells. Long non-coding RNA SNHG14 promotes microglia activation (Qi, X., et al. Neuroscience 348, 98406 (2017), incorporated herein by reference in its entirety). Expressing a mimic of RNA SNHG14 would likely increase the activity of hiPSC-MG to engulf or otherwise kill tumor cells.

Also disclosed herein are methods of manufacturing the induced pluripotent stem cell derived microglia described herein. The methods comprise acquiring induced pluripotent stem cell derived microglia; and introducing one or more of a microglia targeting and activation protein, a modified microglia regulatory protein, an interfering RNA sequence, a microRNA effector, a non-coding RNA effector, a nucleic acid encoding any of the aforementioned, or a combination thereof into the induced pluripotent stem cell derived microglia.

Human induced pluripotent stem cells (hiPSCs) are derived most commonly by introduction of reprogramming factors into blood or skin cells, although cells from other tissues can also be used, leading to formation of cells with the capability of differentiating into virtually every tissue of the body (pluripotency). hiPSCs are different from embryonic stem cells, as embryos are not used to derive hiPSCs. Once hiPSCs are derived, they can be expanded in culture and cryopreserved. Using hiPSCs as a starting point, the cells can be differentiated into hiPSC-MG using a procedure that requires several weeks of in vitro culture. After which, the hiPSC-MG express biomarkers appropriate for MG and exhibit functions appropriate for this cell type, including phagocytosis, uptake of amyloid beta oligomers, and production of cytokines.

Transgenes for one or more of a microglia targeting and activation protein, a modified microglia regulatory protein, an interfering RNA sequence, a microRNA effector, a non-coding RNA effector, or a combination thereof can be prepared by DNA cloning methods and related techniques and introduced into mammalian expression constructs (preparation of transgenes and incorporation into expression constructs are well-developed technologies in modern biology and readily performed using commercially available reagents). Once the expression constructs containing the transgenes are introduced into the hiPSCs via appropriate transfection methods (e.g., utilizing lentiviruses or adenoviruses bioengineered for this purpose, or lipid- or electroporation-based methods) the construct integrates into the host genome. The constructs may also be introduced into the hiPSCs via genomic editing methods including, but not limited to, CRISPR (Clustered Regularly Interspaced Short Palindromic Repeats)-Cas9 in which case the transgene is introduced into a “safe-harbor” location in the genome of the hiPSCs.

The transgenes may be on a vector. In some embodiments, the induced pluripotent stem cell derived microglia comprises one or more transgenes which may be on the same or different vector(s). Vectors can be administered directly to patients (in vivo) or they can be used to manipulate cells in vitro or ex vivo, where the modified cells may be administered to patients. The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Conventional viral and non-viral based gene transfer methods can be used to introduce nucleic acids encoding the transgenes into cells, tissues, or a subject. Such methods can be used to administer nucleic acids encoding the transgenes to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

Additionally, delivery vehicles such as nanoparticle- and lipid-based mRNA or protein delivery systems can be used to introduce one or more of a microglia targeting and activation protein, a modified microglia regulatory protein, an interfering RNA sequence, a microRNA effector, a non-coding RNA effector, a nucleic acid encoding any of the aforementioned, or a combination thereof into the induced pluripotent stem cell derived microglia. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

### 3. Methods

The disclosed the induced pluripotent stem cell derived microglia may be used in various methods, including methods for killing one or more cancer cells, methods of sensitizing cancer cells to phagocytosis and other cell death pathways, and methods of treating cancer in a subject.

In some embodiments, the methods comprise contacting cancer cells with the induced pluripotent stem cell derived microglia described herein. In some embodiments, the cancer cells are brain cancer cells. Such a cancer cell can be from, for example, an astrocytoma, glioblastoma, meningioma, oligodentroglioma, oligoastrocytoma, glioma, ependymoma, spinal cord tumor, ganglioglioma, neurocytoma and medulloblastoma. In some embodiments, the brain cancer cell is from a glioblastoma.

In some embodiments, the cancer cells are in vitro. In some embodiments, the cancer cells are ex vivo. In some embodiments, the cancer cells are in a subject. In some embodiments the subject is a human and the methods comprise administration of the induced pluripotent stem cell derived microglia described herein to the subject.

In some embodiments, the methods comprise administering to the subject a pharmaceutical composition comprising a population of the induced pluripotent stem cell derived microglia described herein. In some embodiments, the subject is a human

The induced pluripotent stem cell derived microglia may be autologous or allogeneic to the subject who is administered the cell. As described herein, the induced pluripotent stem cell derived microglia may be autologous to the subject, e.g., the cells are obtained from the subject in need of the treatment, genetically engineered, and then administered to the same subject. Alternatively, the induced pluripotent stem cell derived microglia are allogeneic cells, e.g., the cells are obtained from a first subject, genetically engineered, and administered to a second subject that is different from the first subject but of the same species (e g , immunological or human leukocyte antigen (HLA)-compatible). In some embodiments, the induced pluripotent stem cell derived microglia are allogeneic cells and have been further genetically engineered to reduced graft-versus-host disease.

In some embodiments the cancer is brain cancer. Any type of brain cancer can be treated with the methods described herein. Brain cancer includes tumor or cancerous cells that originate in the brain (primary brain cancer) as well as tumor or cancerous cells that enter the brain via metastasis from other tissue/cell types (secondary, or metastatic, brain cancer e.g., Her2⁺ metastatic breast cancer). Examples of primary brain cancers include, but are not limited to, gliomas (e.g., astrocytomas, ependymomas, glioblastomas, oligoastrocytomas and oligodendrogliomas), meningiomas, acoustic neuromas, pituitary adenomas, medulloblastomas, and germ cell tumors. Gliomas additionally include recurrent malignant gliomas, high-risk WHO Grade II Astrocytomas, Oligo Astrocytomas, recurrent WHO Grade II Gliomas, newly-diagnosed malignant or intrinsic brain stem gliomas, incompletely resected non-brainstem gliomas, and recurrent unresectable low-grade gliomas. Any cancer can spread to the brain, but non-limiting examples of cancers known to metastasize to the brain include breast cancer, colon cancer, kidney cancer, lung cancers, and melanomas.

In some embodiments, the cancer is glioblastoma. The term “glioblastoma” refers to both primary brain tumors, as well as metastases of the primary brain tumors that may have settled anywhere in the body. The glioblastoma may comprise a newly diagnosed glioblastoma, a recurrent glioblastoma or arise from a lower grade precursor (e.g., astrocytic tumors), a glioblastoma of the proneural type, a glioblastoma of the mesenchymal type, or a glioblastoma of the proliferative type.

The cells may be administered using standard administration techniques, formulations, and/or devices. Provided are formulations and devices, such as syringes and vials, for storage and administration of the compositions.

The induced pluripotent stem cell derived microglia can be administered either alone or in combination with a pharmaceutically acceptable carrier or excipient. The term “pharmaceutically acceptable carrier,” as used herein, means a non-toxic, inert solid, semi-solid or liquid filler, diluent, encapsulating material, or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the disclosed compounds are administered and the form of the composition will dictate the type of carrier to be used.

Compositions in some embodiments are provided as sterile liquid preparations, e.g., isotonic aqueous solutions, suspensions, emulsions, dispersions, or viscous compositions, which may, in some aspects, be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The administration of the compositions contemplated herein may be carried out in any convenient manner, including by injection, ingestion, transfusion, implantation, or transplantation. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection, and infusion. In one embodiment, the compositions contemplated herein are administered to a subject by direct injection into a tumor, lymph node, or a site of surgical resection. In some embodiments, the induced pluripotent stem cell derived microglia described herein are administered to the subject into the brain and CNS concomitantly with resection of a tumor. In some embodiments, the induced pluripotent stem cell derived microglia described herein can be administered into ventricles of the brain via catheter/reservoir delivery systems.

The induced pluripotent stem cell derived microglia described herein may have greater ability to penetrate solid tumors than T cells (which will increase the likelihood for successful treatment) and the induced pluripotent stem cell derived microglia employ different mechanisms (e.g., engulfment) for destroying pathogenic cells and thus are likely to represent a more effective, less dangerous, therapy.

It can generally be stated that a pharmaceutical composition comprising the cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰ or 10¹² cells. In some aspects of the present invention lower numbers of cells, in the range of 10⁶/kilogram (10⁶-10¹¹per patient) may be administered.

Induced pluripotent stem cell derived microglia compositions as described herein may be administered multiple times at dosages within these ranges. The cells may be allogeneic, syngeneic, xenogeneic, or autologous to the patient undergoing therapy.

In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. In some embodiments, one dose of cells can be administered. In some embodiments, the dose of cells can be repeated, e.g., once, twice, or more. In some embodiments, the dose of cells can be administered on, e.g., a daily, weekly, or monthly basis.

A wide range of second therapies may be used in conjunction with the induced pluripotent stem cell derived microglia of the present disclosure. The second therapy may be a therapeutic agent or may be a second therapy not connected to administration of a therapeutic agent. Such second therapies include, but are not limited to, surgery, immunotherapy, radiotherapy, or a chemotherapeutic agent. In some embodiments, the second therapy comprises administration of temozolomide. In some embodiments the second therapy comprises radiotherapy.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. An induced pluripotent stem cell derived microglial cell comprising one or more of:
a) a microglia targeting and activation protein, or a nucleic acid encoding a microglia targeting and activation protein, wherein the microglia targeting and activation protein comprises an extracellular domain comprising a leader sequence, one or more target-recognition domains, a spacer sequence, a transmembrane region, and a cytoplasmic region comprising one or more domains from regulatory proteins that activate microglial cells to engulf or kill target cells;
b) a modified microglia regulatory protein, or a nucleic acid encoding a modified microglia regulatory protein;
c) an interfering RNA sequence, or a nucleic acid encoding an interfering RNA sequence;
d) a microRNA effector, or a nucleic acid encoding a microRNA effector; and
e) a non-coding RNA effector, or a nucleic acid encoding a non-coding RNA effector.

2. The induced pluripotent stem cell derived microglial cell of claim 1, wherein the modified microglia regulatory protein enhances targeting to tumor cells, engulfment or killing of tumor cells, or a combination thereof.

3. The induced pluripotent stem cell derived microglial cell of claim 1, wherein the modified microglia regulatory protein increases antigen presentation of the induced pluripotent stem cell derived microglia.

4. The induced pluripotent stem cell derived microglial cell of claim 1, wherein the interfering RNA sequence is configured to down regulate proteins which results in an increase in recognition, engulfment, and/or killing of tumor cells.

5. The induced pluripotent stem cell derived microglial cell of claim 1, wherein the microRNA effector enhances targeting to tumor cells, engulfment or killing of tumor cells, or a combination thereof.

6. The induced pluripotent stem cell derived microglial cell of claim 1, wherein the microRNA effector is an antagonist of endogenous microRNA effectors.

7. A method of manufacturing the induced pluripotent stem cell derived microglial cell of claim 1, comprising:
acquiring an induced pluripotent stem cell derived microglial cell; and
introducing one or more of a microglia targeting and activation protein, a modified microglia regulatory protein, an interfering RNA sequence, a microRNA effector, a non-coding RNA effector, at least one nucleic acid encoding thereof, or a combination thereof into the induced pluripotent stem cell derived microglial cell.

8. A method of killing one or more cancer cells comprising contacting cancer cells with the induced pluripotent stem cell derived microglial cell of claim 1.

9. The method of claim 8, wherein the cancer cells are brain cancer cells.

10. The method of claim 8, wherein the brain cancer is glioblastoma.

11. The method of claim 8, wherein the one or more cancer cells are in vitro or ex vivo.

12. The method of claim 8, wherein the cancer cells are in a subject.

13. The method of claim 12, wherein the subject is a human.

14. A method of treating cancer in a subject comprising administering to the subject a pharmaceutical composition comprising a population of the induced pluripotent stem cell derived microglial cells of claim 1.

15. The method of claim 14, wherein the cancer is brain cancer.

16. The method of claim 14, wherein the cancer is glioblastoma.

17. The method of claim 14, wherein the subject is a human.

18. Use of the induced pluripotent stem cell derived microglial cell of of claim 1 for killing one or more cancer cells.
